(19) United States
(12) Patent Application Publication
(10) Pub. No.: US 2004/0126272 A1
(43) Pub. Date: Jul. 1, 2004

(54) Near infrared microbial elimination laser system

(76) Inventor: Eric Bornstein, Natick, MA (US)

Correspondence Address: ALTMAN & MARTIN, 6 BEACON ST, STE 600, BOSTON, MA, 02108, US

(21) Appl. No.: 10/649,910

(22) Filed: Aug. 26, 2003

Related U.S. Application Data
(60) Provisional application No. 60/406,493, filed on Aug. 28, 2002.

(51) Int. Cl. A61L 2/00; H01S 5/00

(52) U.S. Cl. 422/22; 372/43

(57) ABSTRACT

A dual wavelength laser in the low infrared electromagnetic spectrum is disclosed for destruction of bacteria via photo-damage optical interactions through direct selective absorption of optical energy by intracellular bacterial chromophores. The dual wavelength (NIMELS) laser includes an optical assembly and all associated components necessary for the housing of two distinct diode laser arrays (870 nm diode array and 930 nm diode array) that can be emitted through an output connector and wavelength multiplexer as necessary. With this preferred design, the dual wavelengths (870 nm and 930 nm) can be emitted singly, or multiplexed together to be conducted along a common optical pathway, or multiple optical pathways, to achieve maximal bacterial elimination.

### CROSS REFERENCE TO RELATED APPLICATIONS

The present application is based upon and claims the filing date of the following pending provisional application: No. 60/406,493, filed Aug. 28, 2002.

### STATEMENT REGARDING FEDERALLY SPONSORED RESEARCH AND DEVELOPMENT

Not Applicable

### BACKGROUND OF THE INVENTION

1. Field of the Invention

The present invention relates to off-site or on-site destruction of bacteria, and, more particularly, to the in-vivo destruction of bacteria by laser energy in medical, dental and veterinary surgical sites, as well as other sites in biological or related systems.

2. Description of the Prior Art

Traditionally solid state diode lasers in the low infrared spectrum (600 nm to 1000 nm) have been used for a variety of purposes in medicine, dentistry, and veterinary science because of their preferential absorption curve to melanin and hemoglobin in biological systems. They rarely have been used for sterilization outside of biological systems.

Because of poor absorption of low infrared diode optical energy in water, its penetration in biological tissue is far greater than that of higher infrared wavelengths. Specifically, diode laser energy can penetrate biological tissue to about 4 centimeters. In contrast, Er:YAG and CO₂ lasers, which have higher water absorption curves, penetrate biological tissue only to about 15 and 75 microns, respectively (10,000 microns=1 cm).

Therefore, with near infrared diode lasers, heat deposition is much deeper in biological tissue, and more therapeutic and beneficial in fighting bacterial infections. However, to prevent unwanted thermal injury to the biological site being irradiated, the radiance (joules/cm2) and/or the exposure time of diode lasers must be kept to a minimum.

For the accomplishment of bacterial cell death with near infrared diode lasers in biological systems, the prior art is characterized by a very narrow therapeutic window. Normal human temperature is 37° C., which corresponds to rapid bacterial growth in most bacterial infections. When radiant energy is applied to a biological system with a near infrared diode laser, the temperature of the irradiated area starts to rise immediately, with each 1° C. rise carrying an injurious biological interaction. At 45° C. there is tissue hyperthermia, at 50° C. there is a reduction in enzyme activity and cell immobility, at 60° C. there is denaturation of proteins and collagen with beginning coagulation, at 80° C. there is a permeabilization of cell membranes, and at 100° C. there is vaporization of water and biological matter. In the event of any significant duration of a temperature above 80° C., (5 to 10 seconds in a local area), irreversible harm to the biological system will result.

To kill bacteria by photothermolysis (heat induced death) in the prior art, a significant temperature increase must occur for a given amount of time in the bacteria containing site. With traditional near infrared diode optical energy, it is desired to destroy bacteria thermally, without causing irreversible heat induced damage to the biological site being treated.

### SUMMARY OF THE INVENTION

The near infrared microbial elimination laser (NIMEL) system, process and product of the present invention utilize a dual wavelength near-infrared solid state diode laser combination in a single housing with a unified control, emitting radiation narrowly at 870 nm and 930 nm. It has been found that these two wavelengths interactively are capable of selectively destroying many forms of bacteria with non-ionizing optical energy and minimal heat deposition. The laser combination of the present invention, which emits these wavelengths simultaneously or alternately, and continuously or intermittently, preferably incorporates at least one ultra-short pulse laser oscillator, composed of titanium-doped sapphire. The system, process and product of the present invention are widely applicable in medical and dental surgery, and in water purification, agriculture, and in emergency and military scenarios.

### BRIEF DESCRIPTION OF THE DRAWINGS

For a fuller understanding of the systems, processes, and products of the present invention, reference is made to the following detailed description, which is to be taken with the accompanying drawings, wherein:

FIG. 1 illustrates the design, partially diagrammatically, of dental instrumentation embodying the laser of the present invention;

FIG. 2a illustrates a dental station incorporating the instrumentation of FIG. 1;

FIG. 2b illustrates details of the control system of the dental station of FIG. 2a;

FIG. 3a shows details of a laser energy delivery head for the instrumentation of FIG. 1;

FIG. 3b shows details of an alternative laser energy delivery head for the instrumentation of FIG. 1;

FIG. 4a shows wavelength division multiplexing details of the laser system of FIG. 1;

FIG. 4b shows further wavelength division multiplexing details of the laser system of FIG. 1;

FIG. 5 illustrates how selected chromophore absorption leads to bacterial cell death pursuant to the present invention;

FIG. 6 illustrates the application of the present invention to periodontal pockets;

FIG. 7 illustrates the application of the present invention to dental scaling instruments;

FIG. 8 illustrates the application of the present invention to root canal procedures;

FIG. 9 illustrates the application of the present invention to ear infections; and

FIG. 10 illustrates the application of the present invention to gangrenous conditions of the fingers and toes.

### DETAILED DESCRIPTION OF THE PREFERRED EMBODIMENT

The present invention is based upon a combination of insights that are derived in part from empirical facts, which include the following.

Most infectious bacteria, when heated, continue growing until their temperature reaches approximately 50° C., whereupon their growth curve slows. At approximately 60° C., bacterial growth comes to an end, except in cases of the hardiest bacterial thermophiles. The range of approximately 60° C. to approximately 80° C. is generally accepted as the time dependent exposure necessary for bacterial death. Hence, in the prior art, there has been a very narrow window of therapeutic opportunity to destroy the bacteria with heat from a traditional near infrared diode laser (60° C. to 80° C.) without causing irreversible heat induced damage (more than 5 sec) to the biological site being treated.

The dual wavelength, solid state, near-infrared diode laser system of the present invention is specifically designed for bacterial destruction with minimal heat deposition in the site being irradiated. It has been found that the wavelength combination of the present invention is capable of destroying bacterial cells as a result of the interaction of a toxic singlet oxygen reaction that is generated by the absorption of laser energy selectively in intracellular bacterial chromophores. These chromophores happen to be specific to wavelengths that narrowly approximate 870 nm and 930 nm in the near infrared spectrum.

Without the significant heat deposition normally associated in the previous art with continuous wave or pulsed near infrared diode lasers, bacteria can be selectively destroyed while minimizing unwanted hyperthermia of the irradiated tissues and the surrounding region. The point where the system, process and product of the present invention depart from conventional thermal bacterial destruction is based on research conducted with the technology of so-called optical cell trapping and optical tweezers.

Optical tweezers are low infrared based optical traps (created for cell biology), which simply use infrared laser beams of very low power to hold and study single cells of various prokaryotic and eukaryotic species while keeping them alive and functional under a microscope. When this procedure is effected with low infrared laser energy, intense heat deposition occurs. To accomplish the goal of “holding” a single cell in place without killing it through thermolysis, the laser energy must be reduced to under 100 milliwatts of energy. Thereby, the bacteria may be kept alive for a five minute period. In an elegant study using a tunable Ti:Sapphire laser, Neuman (Biophysical Journal, Vol. 77, November 1999) found that, even with this very low laser output to rule out direct heating (thermolysis) as the source of bacterial death, there are two distinct wavelengths that cannot be used successfully for optical traps because of their lethal affect on E-coli bacteria. These wavelengths are 870 nm and 930 nm.

Neuman found that the two wavelengths, 870 nm and 930 nm (in contrast to all others in the low infrared spectrum), are not transparent to the bacteria being studied. He postulated that the two wavelengths probably interact with a linear one photon process mediated through absorption of one or more specific intracellular bacterial chromophores or pigments. This one photon process of photodamage (not thermal damage) to the bacteria, he further concluded, implies a critical role for a short acting singlet oxygen species, or a reactive oxygen species as the culprit in the cellular damage pathway. (This may be a common damage pathway for eukaryotic systems, but must be further studied as the eukaryotic cell line studied (chinese hamster hela ovary cells) are fragile in nature compared to many other eukaryotic cells.) Accordingly, the system, process and product of the the present invention are characterized by the following general considerations.

The present invention provides a dual wavelength diode laser combination to be used for bacterial destruction with minimal heat deposition in human medicine and dentistry, veterinary medicine, water purification, agriculture, and military scenarios.

If used in any medical, biological, military or industrial system, the diode oscillators can be used singly or multiplexed together to effect maximal bacterial death rates in the site being irradiated.

In various embodiments, the energies from both diode laser oscillators preferably are conducted, either singly or multiplexed, along a common optical pathway to effect maximal bacterial death rates in the site being irradiated.

In certain alternative embodiments, the energies from both diode laser oscillators are delivered separately, simultaneously or alternately through multiple optical pathways.

In accordance with the present invention, it is critical that the laser wavelengths selected as approximating 870 nm and 930 nm, respectively lie within the wavelength ranges of (a) 865 nm to 875 nm and (b) 925 nm to 935 nm.

Instead of avoiding the 870 nm and 930 nm wavelengths as suggested in the prior art by optical tweezer procedures, the laser system and process of the present invention selectively combines them. With less heat deposition in the site being irradiated, a much enlarged therapeutic window of opportunity is available to the laser operator. In essence, the combined wavelengths of the present invention use less energy than do prior art procedures to effect bacterial destruction, i.e. the optical energy used in the present invention is less than the thermal energy used in the prior art.

The medical, dental or veterinary applications of the dual wavelength combination of the present invention include, but are not limited to, coagulation, tissue vaporization, tissue cutting, selected photodynamic therapy, and interstitial thermal-therapy.

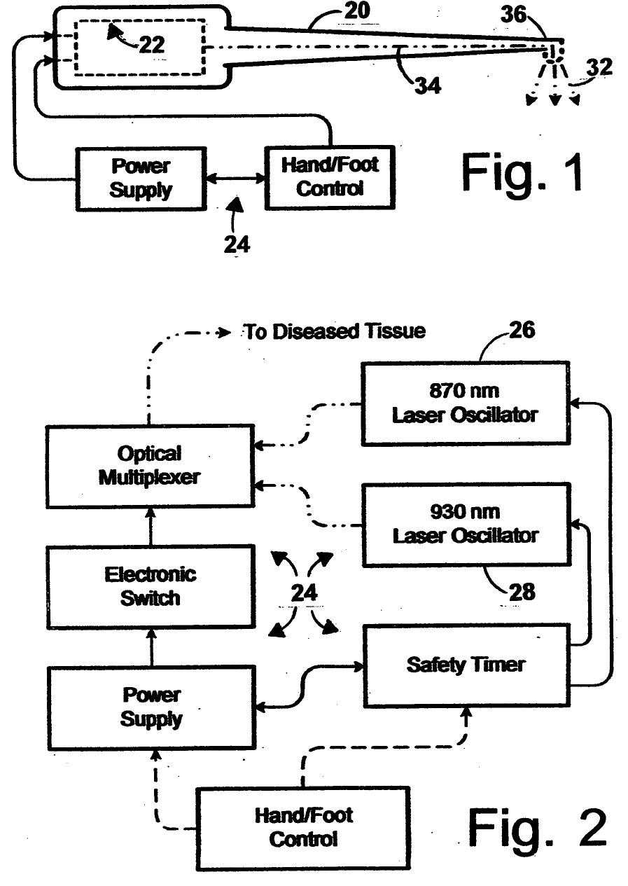

### FIGS. 1 to 5—The Dual Wavelength System

A laser system for destroying bacteria in a bacterial dental site is shown in FIGS. 1-5 as comprising a housing 20 and a control 22, 24. Within the housing is a laser oscillator sub-system 26, 28 for causing the selective emission of radiation 30 in a first wavelength range of 865 nm to 875 nm, and the selective emission of radiation 32 in a second wavelength range of 865 nm to 875 nm. The radiation is propagated through an optical channel 34 to a head 36 for enabling delivery of the radiation through the optical channel to a bacterial site.

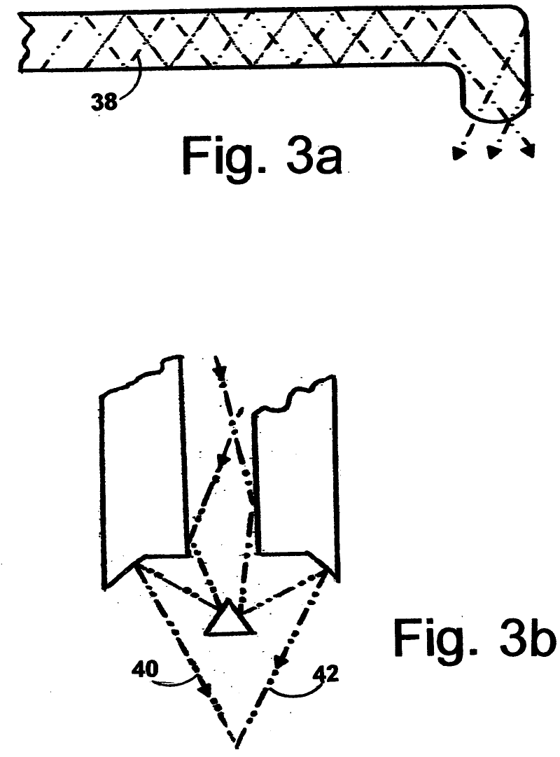

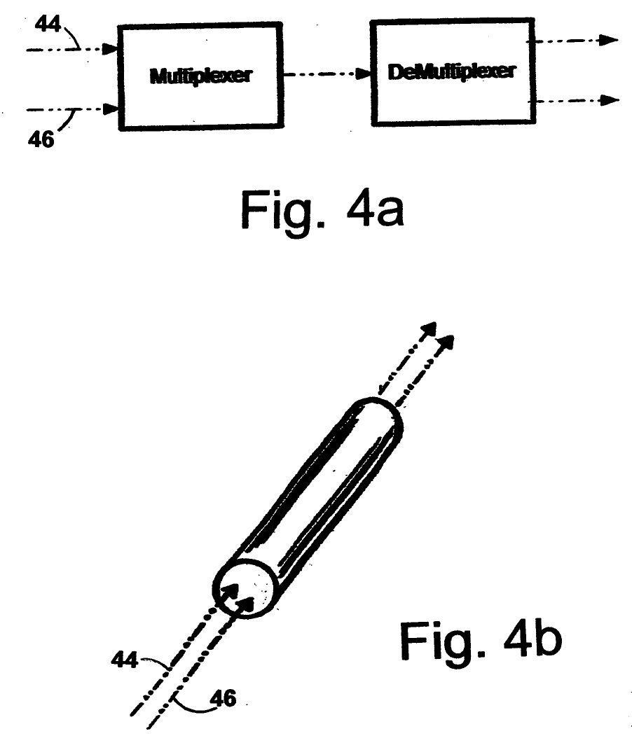

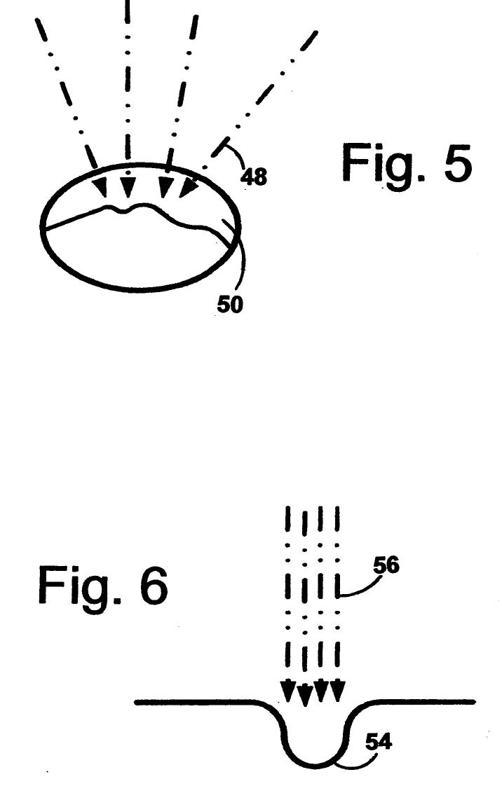

In various delivery systems: the transmission is simultaneous as shown at 38 in FIG. 3a, alternate as shown at 40, 42 in FIG. 3b, and/or multiplexed as shown at 44, 46 in FIGS. 4a and 4b. As shown in FIG. 5, the two wavelengths generate a chromophore 48 from the bacterial site and cooperate with the chromophore at 50 to destroy bacteria in the bacterial site.

### FIG. 6—Periodontal Pocket Therapy

FIG. 6 illustrates a system 52 embodying the present invention that is designed for use in the therapeutic treatment of a deleterious ecological niche 54 known as a periodontal pocket. Laser energy wavelengths of 870 nm and 930 nm is shown as being emitted from a desktop laser and dispersed through the distal end of an optical fiber within the periodontal pocket to achieve bacterial elimination. The dual laser construction is intended to limit the use of antibiotics and conventional periodontal surgery to destroy bacteria in a periodontal pocket.

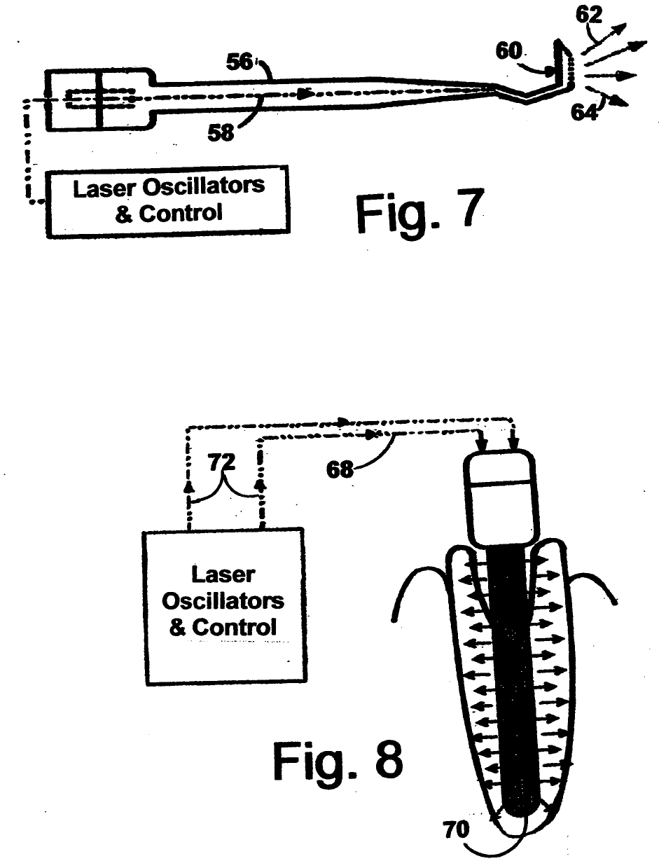

### FIG. 7—Laser Augmented Dental Scaling

FIG. 7 illustrates a system 56 embodying the present invention, which is designed to channel the dual wavelength energy of the present invention through the hollow axis 58 of a laser augmented periodontal scaling instrument 60 having scaling edges 62, 64 to effect bacterial elimination while mechanically debriding the root surface of a tooth. This dual wavelength system is intended to limit the necessity of antibiotics in periodontal surgery.

### FIG. 8—Laser Augmented Root Canal Therapy

FIG. 8 illustrates a system 68 by which a laser embodying the present invention is designed for use in the therapeutic treatment of bacteria in the root canal of a tooth being treated. The objective is to provide targeted energy for the infected root canal space within a tooth to achieve bacterial elimination within the dentinal tubules. As shown, dual wavelength energy of the present invention is dispersed through a laser augmented root canal interstitial thermal therapy tip 70, connected to an optical fiber 72 to achieve the bacterial elimination. This system is intended to limit the need for antibiotics for root canal therapy.

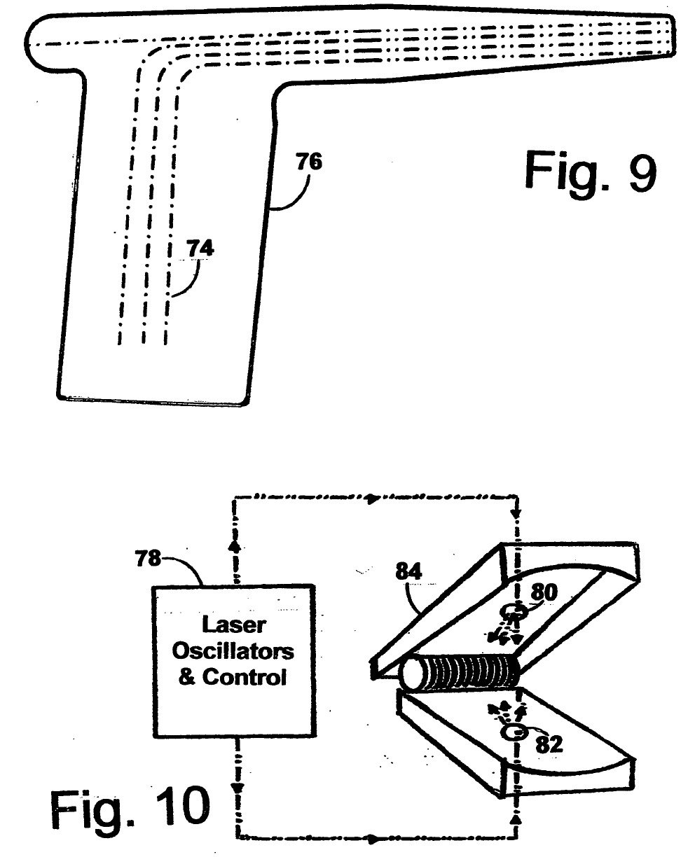

### FIG. 9—Laser Augmented Otoscope

FIG. 9 shows the therapeutic use of dual wavelength energy 74 in accordance with the present invention as an adjunct for curing otitis media (ear infections). As shown, the dual wavelength energy 74 is channeled at 76 through an otoscope having an optical channel for conduction of the energy. This allows the practitioner under direct visualization, to irradiate the inner ear drum and canal dual laser energy to effect bacterial elimination without thermal tissue destruction.

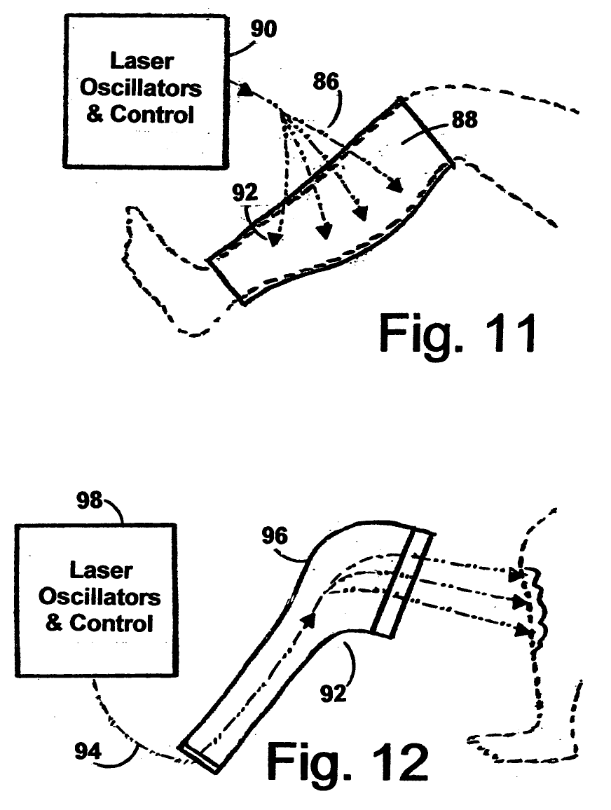

### FIG. 10—Treatment for Gangrenous Fingers and Toes

FIG. 10 shows a system 78 embodying the present invention for use as an adjunct to treat infected and gangrenous fingers and toes in diabetic patients. In the preferred embodiment for this approach, the dual wavelength is dispersed through dual apertures 80 and 82 in a plastic clip 84. The clip is intended to be clamped on the diseased digit (finger or toe) of a patient and to bathe an infected area of the digit for a defined period at a defined power to effect bacterial elimination without detrimental heat deposition.

### FIG. 11—Laser Augmented Therapeutic Stocking

FIG. 11 shows a system 86 embodying the present invention for use as an adjunct for the treatment of a limb that is infected with cellulites and/or necrotizing fasciitis. As shown, dual wavelength energy of the present invention is dispersed through a fiber optic illuminating fabric 88 with ingress from a dual wavelength source 90 and egress 92 in communication with the limb. This fabric is in the shape of a stocking that is wrapped around an infected area, to disperse the dual wavelength optical energy to the limb being treated to eradicate bacteria.

### FIG. 12—Therapeutic Wand

FIG. 12 shows a system 92 for applying dual wavelength energy broadly in accordance with the present invention for bacterial elimination of an infected wound or surgical site. The dual wavelength energy is dispersed through a channel 94 in an elongated wand 96 that is directed orthogonally toward the infected wound to optically accomplish bacterial elimination. It is intended that instrument be used in a hospital setting or in conjunction with a battery powered field pack 98.

### Operation

In operation, each of the illustrated embodiments is capable of generating continuous wave or pulsed laser energy independently or at the same time depending on the parameters set by the operator. To this laser is connected a hollow wave guide or a suitable fiber optic delivery system. This system is capable of generating from 100 mw up to 20 watts of laser output from each wavelength independently or a total of 200 mw up to 40 watts together depending on the parameters set by the operator. By using the bacterias own chromophores, the system produces maximum lethal effects on the bacteria with minimal heat deposition.

It specifically illustrated the selected bacterial intracellular chromophore absorption of either or both laser energies singly or simultaneously, which leads to bacterial cell death by creating lethal photo-damage to the bacteria independently of the normal mode of thermal damage normally seen with other wavelengths of near infrared solid state diode lasers. Applications include a significant positive impact on the fields of human and veterinary medicine and dentistry, laboratory biology and microbiology, food service, and any other area needing bacterial control without the unwanted side effects of ionizing radiation, ultraviolet light, and heat deposition. The purpose of such radiant exposure in the prior art, in various embodiments, are ablation of tissue, vaporization of tissue, coagulation of a surgical area, photochemical interactions, and bacterial death by thermolysis of bacterial cells. Heat flow in this system, which is the transfer of thermal energy through the tissue, is generally measured in joules. Infrared radiation is known as “heat radiation” because it directly generates heat in an absorptive medium.

What is claimed is:

1. A laser system for destroying bacteria in a bacterial locale, said system comprising:
(a) a housing and a control;
(b) a laser oscillator sub-system within said housing for causing the selective emission under said control of first radiation in a first wavelength range of 865 nm to 875 nm and the selective emission under said control of second radiation at a second wavelength range of 865 nm to 875 nm;
(d) an optical channel for transmission of said first radiation and said second radiation from said laser oscillator sub-system; and
(c) a head for enabling delivery of said first radiation and said second radiation from said laser oscillator sub-system through said optical channel to the site of said bacterial locale;
(d) said first radiation and said second radiation being adapted to generate a chromophore from said bacterial locale and being adapted to cooperate with said chromophore to destroy bacteria in said bacterial locale.

2. The laser system of claim 1, wherein said transmission is simultaneous.

3. The laser system of claim 1, wherein said transmission is alternate.

4. The laser system of claim 1, wherein said transmission is multiplexed;

5. The laser system of claim 1, wherein said head includes an optical egress for said first radiation and said second radiation, and a scaling instrument.

6. The laser system of claim 1, wherein said head includes an optical egress having a frosted tip.

7. The laser system of claim 1, wherein said head includes an optical egress and an otoscope.

8. The laser system of claim 1, wherein said head includes a digit clip and an optical egress therefrom.

9. The laser system of claim 1, wherein said head includes a stocking having an optical ingress from said laser oscillator and an optical egress to the inner surface of said stocking.

10. The laser system of claim 1, wherein said head includes a handle and an optical egress extending therefrom.

11. A laser system for destroying bacteria in a bacterial locale, said system comprising:
(a) a housing and a control;
(b) a laser oscillator sub-system within said housing for causing the selective emission under said control of first radiation narrowly at a first wavelength range of 870 nm and the selective emission under said control of second radiation at a second wavelength range of 930 nm; and
(c) a head for delivering said first radiation and said second radiation from said laser oscillator sub-system to the site of said bacterial locale;
(d) said first radiation and said second radiation being adapted to generate a chromophore from said bacterial locale and being adapted to cooperate with said chromophore to destroy bacteria in said bacterial locale.

12. The laser system of claim 11, wherein said transmission is simultaneous.

13. The laser system of claim 11, wherein said transmission is alternate.

14. The laser system of claim 11, wherein said transmission is multiplexed;

15. The laser system of claim 11, wherein said head includes an optical egress for said first radiation and said second radiation, and a scaling instrument.

16. The laser system of claim 11, wherein said head includes an optical egress having a frosted tip.

17. The laser system of claim 11, wherein said head includes an optical egress and an otoscope.

18. The laser system of claim 11, wherein said head includes a digit clip and an optical egress therefrom.

19. The laser system of claim 11, wherein said head includes a stocking having an optical ingress from said laser oscillator and an optical egress to the inner surface of said stocking.

20. The laser system of claim 11, wherein said head includes a handle and an optical egress extending therefrom.

21. A process for destroying bacteria in a bacterial locale, said process comprising:
(a) energizing a laser to cause the selective emission of first radiation in a first wavelength range of 865 nm to 875 nm and the selective emission of second radiation at a second wavelength range of 865 nm to 875 nm;
(b) establishing a path for the transmission of said first radiation and said second radiation from said laser oscillator sub-system; and
(c) enabling delivery of said first radiation and said second radiation from said laser oscillator sub-system through said optical channel to the site of said bacterial locale;
(d) said first radiation and said second radiation generating a chromophore from said bacterial locale and cooperating with said chromophore to destroy bacteria in said bacterial locale.

22. A process for destroying bacteria in a bacterial locale, said process comprising:
(a) energizing a laser to cause the selective emission of first radiation in the selected wavelength of 870 nm and the selective emission of second radiation in the selective wavelength range of 930 nm;
(b) establishing a path for the transmission of said first radiation and said second radiation from said laser oscillator sub-system; and
(c) enabling delivery of said first radiation and said second radiation from said laser oscillator sub-system through said optical channel to the site of said bacterial locale;
(d) said first radiation and said second radiation generating a chromophore from said bacterial locale and cooperating with said chromophore to destroy bacteria in said bacterial locale.
